(19) United States
(12) Patent Application Publication
(10) Pub. No.: US 2019/0223718 A1
(43) Pub. Date: Jul. 25, 2019

(54) EYE EXAMINING DEVICE

(71) Applicant: HAAG-STREIT AG, Koeniz (CH)

(72) Inventors: Joerg Breitenstein, Zollikofen (CH); Frank Zumkehr, Zollikofen (CH); Claudio Dellagiacoma, Bern (CH)

(73) Assignee: HAAG-STREIT AG, Koeniz (CH)

(21) Appl. No.: 16/256,798

(22) Filed: Jan. 24, 2019

(30) Foreign Application Priority Data
Jan. 25, 2018 (CH) 00088/18

(51) Int. Cl. A61B 3/135; A61B 3/14

(52) U.S. Cl. CPC A61B 3/135; H04N 5/235; A61B 3/14

(57) ABSTRACT

A device (1) for examining an eye, in particular a slit lamp, comprises an image recording unit (220) with at least one first sensor (241.1) in a first beam path and a second sensor (241.2) in a second beam path, the device also comprising in the first beam path a first objective with a first optical axis, which has a fixed magnification and is arranged as fixed in the first beam path.

### TECHNICAL FIELD

The invention relates to a device for examining an eye, in particular a slit lamp, comprising an image recording unit with at least one first sensor in a first beam path and a second sensor in a second beam path. The invention also relates to a method for determining image data with a device for examining an eye.

### PRIOR ART

Slit lamp microscopes are opthalmological examining devices with which the eyes can be monoscopically or stereoscopically examined. For the stereoscopic viewing of an eye, known slit lamp microscopes have an optical unit for producing two images of an eye and two eyepieces for the stereoscopic viewing of the images and also a lighting unit. The lighting unit is arranged on a vertically running branch of a holding unit. The eye to be viewed can be positioned in an approximately horizontally running plane on one side of the holding unit. The lighting unit comprises an incandescent lamp, LEDs or similar lighting means for slit lighting.

EP 2 446 812 BI (Haag-Streit) discloses a slit lamp, which comprises two image sensors for the electronic recording of two images in order to achieve a three-dimensional presentation, it also being possible for more than two image sensors to be provided. Furthermore, image sensors with different spectral ranges can be provided. The electronically recorded image data can be processed, in particular optimized, abstracted, etc, during the presentation.

US 2010/201799 A (Zeiss) relates to an ophthalmological device for achieving images with an expanded dynamic range. The device comprises at least one beam splitter and at least two image sensors, and also a slit lamp or a fundus camera. This allows multiple images with an identical scene to be recorded and put together to form an overall image. The beam splitter has an asymmetrical splitting setting.

DE102010014114 (Zeiss) relates to an application of an image recording unit of an ophthalmological device for adjusting and measuring tasks. The image recording device comprises a digital camera with high resolution and a digital zoom function.

The known devices for examining an eye have the disadvantage that they are complex and expensive to produce.

### DESCRIPTION OF THE INVENTION

The invention addresses the problem of providing a device belonging, to the technical field mentioned at the beginning for examining an eye that is of a particularly low cost and robust form.

The solution to the problem is defined by the features of claim 1. According to the invention, the device also comprises in the first beam path a first objective with a first optical axis, which has a fixed magnification and is arranged as fixed in the first beam path.

Optomechanical devices are very expensive to produce, because they have to be extremely precise and robust. The fact that the objective has a fixed magnification means that the number of moving parts in the device can be reduced, whereby the device as a whole can be produced at lower cost.

Hereinafter, the expression “a and/or b” is understood as meaning in each case a nonempty subset of the elements a and b, that is to say either a or b or (a and b).

The expression “arranged as fixed in the beam path” is understood in the present case as meaning that the objective stays in the beam path—although it is clear to a person skilled in the art that the objective defines the beam path, or plays a part in defining the beam path. Consequently, the fixed arrangement in the beam path should be understood as meaning that the device is not formed in such a way that the objective in the beam path cannot be replaced by another objective. However, one or more of the objectives may be pivotably or displaceably formed, the beam path also being changed by corresponding pivoting or displacement of the objective.

The device is preferably formed as a slit lamp, whereby in a preferred application a front portion of the eye can be examined. However, it remains clear to a person skilled in the art that the invention can also be transferred to related devices; in particular, the invention may also be realized in the case of a fundus camera or the like. The expression “slit lamp” should be understood as meaning a device for the stereoscopic examination of an eye which comprises a device for slit lighting. Such devices are sufficiently well known to a person skilled in the art.

The image recording unit comprises at least a first sensor and a second sensor. The sensors are consequently preferably suitable for recording an image. It is not absolutely necessary here for the sensor to operate in the visible spectral range (see below). The sensor may under some circumstances be made up of multiple individual sensors. Hereinafter, the sensor is understood as meaning in each case an individual sensor which is assigned to an objective or a sensor consisting of multiple individual sensors which is likewise assigned to an objective. Particularly preferably, the first sensor and/or the second sensor is a digital sensor. In particular because no optomechanical magnification is provided in the preferred embodiment apart from possibly present multiple objectives with different fixed magnification, digital image magnifications can be performed particularly easily with the digital sensors, in particular using known techniques (interpolation, etc.). The fact that a first sensor and a second sensor are provided means that, with suitable alignment of the two sensors, a stereoscopic recording is possible. Moreover, image data of the same segment of an object from multiple sensors is computationally combined to form a single image. This allows the quality of the image to be improved considerably in comparison with the individual images. It is in this way possible with multiple individual sensors, each having multiple objectives, to simulate a sensor which has a higher resolution than the individual sensors. This allows a relatively low-cost device to be achieved in relation to the achievable resolution.

In the first beam path there is the first sensor and in a second beam path there is the second sensor. The expression “beam path” is defined by those rays of light that reach the sensor from the object that is being observed. The device according to the invention may comprise in addition to the sensor a beam splitter for an eyepiece. Unless otherwise stated, the rays of light which in this case reach the eyepiece from the object are not assigned to the beam path. In the preferred embodiment, however, the device does not comprise any eyepieces in the classical sense, but at most electronic eyepieces or the like that are provided with screens. The beam path is typically a polygonal traverse, along which a light beam passes from the object through the optical elements (lenses, mirrors, etc.) to the sensor. Depending on the form of the optical elements, the beam path may however also be formed as a straight line or comprise curves.

The expression objective is understood hereinafter as meaning the optical element of the device that is arranged in the beam path adjacent to the observed optical element. That is to say that the objective lies between the image and the object.

According to the invention, the first objective has a fixed magnification. Consequently, the first objective has a fixed focal length. Objectives with a fixed focal length typically allow recordings with better image quality than zoom lenses, because they are of a simpler construction with fewer elements, or fewer movable elements.

In principle, it would be conceivable to provide the first objective with the fixed focal length in an exchangeable manner, so that different objectives could be used to achieve different focal lengths during the recordings. The invention, however, dispenses with this exchangeability, so that the first objective with the fixed magnification is arranged as fixed in the first beam path. Movable parts generally represent a source of errors for the device. In particular, if the movable parts are located as optical elements in the beam path, or can be inserted into the beam path, even minimal deviations from the desired position can lead to imaging errors. Arranging the first objective as fixed in the first beam path means that a particularly robust and precise device is achieved. Making it possible to dispense with complex optomechanical components also allows the device as a whole to be less costly.

Particularly preferably, the device is formed without an eyepiece for analog viewing of the object. The device is consequently preferably designed exclusively for digital presentation of the image data recorded by the image sensors. Because the image data are digitized, it is possible to use for viewing the image data a presentation unit, in particular an electronic display or the like, that can be formed as positionable independently of the optical unit, in particular independently of the eyepieces. This allows a particularly ergonomic posture to be achieved for the user during the application. For this purpose, the device may have one or more electronic screens or displays. The means for presenting the image data do not necessarily have to be comprised by the device itself. It is also possible for the device merely to be designed for recording the image data, while the presentation of the image data is performed by a separate unit. This allows the device as a whole to be simplified further, in particular since the eyepieces and their lens systems are expensive to produce.

In variants it is possible, for example by using a beam splitter, for the device to comprise an eyepiece in one beam path or in both beam paths, whereby the object can be viewed in the classical way in an analog form (i.e. not digitized).

Preferably, the device has a focal plane changeover, allowing the changeover between two or more focal planes. Preferably, the device comprises such a focal plane changeover for each sensor.

In variants, a continuously variable setting of the focal plane may also be provided. Alternatively, it is also possible to dispense with the focal plane changeover. In this case, for example the device may be movable in relation to the object in such a way that the focal plane can be set. For this purpose, either the object (for example a chin and forehead support in the case of a split lamp) or the device may be movable.

Preferably, the first objective comprises the first sensor. In this way, a particularly compact construction of the device is achieved. In particular, in this way an alignability of the objective with an object can be realized in an easy way. The device preferably also comprises a second objective with a second optical axis, the second objective comprising the second sensor and in particular having a fixed magnification and being arranged as fixed in the second beam path. This has the overall effect of achieving a low-cost construction of a device with which an eye can be viewed stereoscopically. The two objectives do not necessarily have to be used for achieving stereoscopic recordings. The two objectives with the sensors may also be used for achieving other effects; in particular, the two sensors may be used for example for recording different regions of the eye or covering different spectra (see below).

In variants, the sensor may also be arranged downstream of the objective in the beam path, so that for example further optical elements can be arranged between the objective and the sensor.

Preferably, the first sensor and the second sensor differ in a recording spectrum. This allows greater spectral ranges to be covered with great sensitivity.

In variants, the first sensor and the second sensor may also have the same recording spectrum. In this case, a single image with increased resolution and/or sharpness can be achieved with the overlapping image data of the two sensors. The two sensors may in particular also have the same recording spectrum, but with a different resolution.

Preferably, the first sensor is formed as a color sensor, in particular for example as a RGB or white-light sensor and the second sensor is formed as a black-and-white sensor or as an IR sensor. The black-and-white sensor has the advantage over the color sensor that higher resolutions are possible with a comparable number of pixels. On account of the color filters in the case of the color sensors, in particular the Bayer filter, the resolution is generally reduced by approximately a factor of 2. On the other hand, the color sensor has the advantage that the color aspects, which may be very helpful in the examination of an eye, can be recorded. The combination of a color sensor as the first sensor with a black-and-white sensor as the second sensor thus allows the advantages of the two sensors to be combined.

Preferably, the image data of a first sensor, formed as a black-and-white sensor with a first resolution, are computationally combined with image data of a second sensor, formed as a color sensor, to form a single colored image and/or to form a sequence of colored images, the second sensor having a resolution that is in particular lower than the resolution of the first sensor. That the second sensor, that is to say the color sensor, has a lower resolution than the black-and-white sensor makes it possible to provide a particularly low-cost method for obtaining high-resolution but nevertheless color image data.

The combination of a sensor operating in the visible range with an IR sensor also allows for example inflammations in the eye to be detected, since inflamed areas generally have an increased temperature.

Finally, sensors with other spectral ranges may also be used, in particular sensors with narrow spectral ranges, which are thus also used in chemical analytics.

Alternatively, one of the sensors may also be formed as a UV sensor. Consequently, a UV-sensitive marker in the object could be observed. Further sensor types and applications for the sensor types are known to a person skilled in the art.

Preferably, the device has at least one third sensor in a third beam path. With the third sensor, the application area of the device can be extended further. The third sensor may for example be provided for creating an overview image, while the first and second sensors may record an image segment of the overview image.

The third sensor may also differ from one or both of the other sensors in a recording spectrum. In a particularly preferred embodiment, the second sensor is formed as a color sensor and the third sensor is formed as an IR sensor, while in particular the first sensor is formed for example as a black-and-white sensor. In this variant, the advantages of the three sensor types can be combined. Alternatively, all of the sensors may also have the same recording spectrum. For example, all of the sensors may be formed as color sensors or as black-and-white sensors.

The third sensor is preferably comprised by a third objective, arranged as fixed in the third beam path, and preferably has a fixed magnification. In this way, once again a particularly robust and low-cost device is achieved. If the third sensor is formed for creating, an overview image, the third objective may have a smaller magnification than the first and second objectives. In this case, the first and second objectives may for example comprise a color sensor and have the same magnification, so that they can be used in the conventional way for stereoscopic viewing of the object, in particular an eye.

In variants, it is also possible to dispense with the third sensor. Furthermore, is also possible to use more than three sensors.

Preferably, a first magnification factor of the first objective is less than a second magnification factor of the second objective. This allows a greater image recording range to be covered by the first sensor than by the second sensor. In this embodiment for example an overview image can be created with the first sensor, while a segment of the overview image can be recorded in greater resolution with the second sensor. In the method, a region of the image recorded with the first sensor may be zoomed onto until the resolution of the image segment of the region of the image is no longer sufficient. From this point in time, it is possible to change from the first sensor to the second sensor, in order to be able to view the image segment with greater magnification in a highly resolved form. This allows low-cost sensors to be used for nevertheless achieving image segments with high resolution.

In variants, the first objective and the second objective may also have the same magnification. In particular if an objective with great magnification is dispensed with in all of the beam paths, a high resolution can nevertheless be achieved by the use of correspondingly highly resolving (and consequently typically more expensive) sensors.

Preferably, an image recording region in a focal plane of the first beam path overlaps at least partially with an image recording region in a focal plane of the second beam path. The expression image recording region is understood as meaning that region in the focal plane that is given by the image angles of the objective, i.e. it is the region that can be imaged by the objective. The partial overlapping allows a greater image recording region to be covered by the multiple sensors. The overlapping of the image recording region also achieves the effect that the image data are available in duplicate in the overlapping region, so that between the individual recordings a transition can be optimally calculated. This allows reflexes and the like to be eliminated digitally on the basis of the redundant information.

In one embodiment, four objectives with an image sensor in each case may be provided for example, the image sensors covering a rectangular region in such a way that the overlapping forms a cross shape.

In variants, the sensors may also be formed and aligned in such a way that different image recording regions are covered in each case. Thus, for example four objectives with an image sensor in each case may be aligned in such a way that an image recording region without overlapping is obtained.

Preferably, the image region of the focal plane of the second beam path lies within the image region of the focal plane of the first beam path. This allows an overview image to be recorded with the first sensor and a segment of the overview image to be recorded with the second sensor. The technical conversion can be achieved in various ways. In the first variant, the first sensor and the second sensor may be of different sizes. In a second variant, the same sensor in each case may be inserted into an objective with different magnification. Furthermore, the same objectives with the same sensors may be arranged at different distances from the object. Further variations are known to a person skilled in the art; in particular, the above features may also be combined, so that for example different objectives are arranged at different distances from the object.

In variants, the focal planes of the first beam path may also coincide with the focal plane of the second beam path.

Preferably, an angle between the first optical axis and the second optical axis can be set. Preferably, the optical axes are spatially adjustable. For this purpose, an objective may for example have two pivot axes at right angles to one another. This allows the focal planes of the first objective and the second objective to be moved in relation to one another. In particular when the first objective is used for creating an overview image, this has the advantage that for example with the second objective an image segment can be chosen and varied. Particularly preferably, at least one objective with a possibly greater magnification is formed as movable, while an objective for creating an overview image does not necessarily have to be formed as movable, in particular if the objective for creating the overview image completely covers the image recording region in the fixed positioning. Alternatively, multiple objectives may also be formed as movable.

In variants, an angle between the optical axes of the first objective and the second objective may also be fixed.

Preferably, the first optical axis and the second optical axis can be set as parallel. This allows a particularly large image recording region to be covered with the first sensor and the second sensor. Preferably, the third optical axis can also be aligned in parallel with one or both of the other optical axes.

Alternatively, it is also possible to dispense with the parallel alignment of the first optical axis and the second optical axis.

Preferably, a diaphragm and/or an optical filter, which can in particular be actuated in a motorized manner, are arranged in the first optical axis and in the second optical axis. The diaphragm is preferably formed as an adjustable diaphragm, whereby magnification-independent control of the depth of focus can be achieved. Alternatively, it is also possible to dispense with the adjustability of the diaphragm. The filter is preferably a color filter or multiple selectable color filters. Preferably, the color filter can be actuated in a motorized manner, which is to say that it can be extended into and retracted from one or more of the beam paths. This allows the handling of the device to be simplified.

In a particularly preferred embodiment, the imaging of the slit is produced by means of a projection method, preferably by means of DLP. This makes it possible to dispense with mechanical parts, whereby the device can once again be formed at lower cost.

In variants it is also possible to dispense with the filter and respectively also with the motorized actuability. Slit lighting can be produced by means of a conventional, mechanical diaphragm.

The device may in particular also comprise a control unit, whereby the slit form and/or the filter selection can also be automatically chosen or respectively adapted and optimized on the basis of the data measured.

Preferably, the slit lighting and the sensors can be synchronized in such a way that the slit lighting is activated at the same time as the sensors, in particular during the exposure time. In particular when viewing an eye, this allows undesired reactions and reflexes of the eye, such as for example the opening of the pupil, etc., during the recording of the image to be avoided. Consequently, in the method the lighting and the sensors are preferably activated at the same. In the present case, the expression “at the same time” permits slight temporal deviations that do not adversely affect the recording of the image. Thus, for example, the lighting may be activated in the range of milliseconds before the activation of the sensors in order to avoid the sensors being activated before the lighting as a result of the given error tolerances. Similarly, the lighting may be activated at any time slightly after the deactivation of the image sensors. This ensures that the lighting is active during the exposure time of the sensors.

In variants, it is also possible to dispense with the synchronizability of the lighting and the sensors.

In a method for determining image data, the following steps are carried out:
recording of image data of an object by the first sensor and the second sensor, in particular from different perspectives;
processing image data of the first sensor and the second sensor, in particular to form a single image and/or a sequence of images or respectively to form a single image segment and/or a sequence of image segments.

The processing of the image data of the first sensor and the second sensor to form a single image may have the effect for example of improving the quality of the image. Thus, for example, the image data of two image sensors may be computationally combined to form an image with improved image quality in comparison with the individual images. The two sensors and/or the objectives may however also differ in a property, in order to be able with the computationally combined image to benefit from the advantages of the different properties, which under some circumstances may rule one another out in the case of a single image sensor.

Preferably, the image data of the object are recorded by the first sensor and the second sensor at the same time. In particular in the case of moving objects, such as for example in the case of an eye, it is thereby possible to achieve image data that show the object in the same state and in the same alignment. This allows the image data of the individual sensors to be computationally combined in an easy way to form a single image, since movement artifacts do not have to be taken into account, or scarcely.

In variants, the image data can also be recorded with the sensors temporally one after the other.

Preferably, the recording conditions of the first sensor and the second sensor differ, in particular in one of the following features:

a. exposure time;
b. diaphragm setting;
c. filter setting;
d. recording spectrum.

A variation of the recording properties of the first sensor and the second sensor allows image data that have higher qualities in different regions to be obtained. In this way it is possible in particular to perform settings that would conflict with one another in the case of a single sensor.

For example, a long exposure time may be chosen for the first sensor, in order to achieve a high image quality, while a short exposure time is chosen with the second sensor, in order as far as possible to achieve no movement artifacts, and consequently a sharp image. With a combination of the image data of the first sensor and the second sensor, the advantages of the two settings can thus be combined to form a single image.

In a further example, a large diaphragm opening may be chosen for the first sensor, in order to obtain a great incident of light on the first sensor, while a smaller diaphragm opening is chosen for the second sensor, in order as far as possible to achieve a great depth of field. Once again, by processing the image data, the advantages of the respective settings can be combined in a single image.

Furthermore, different filters, for example color filters, may be used in the case of the two sensors, it being possible for the advantages of the individual filters to be combined in a processing of the image data to form a single image. For example, contrasts can be specifically changed by a suitable choice of color filters.

By choosing different recording spectra for the two image sensors, once again different contrasts can be obtained. The image data can once again be processed to form a single image, in which the advantages of the individual recording spectra can be combined in one image.

Further recording conditions that can be chosen differently in the case of the first sensor and the second sensor are known to a person skilled in the art. The advantages are obtained in particular in the case of a substantially simultaneous recording of the same image segment. However, it is clear to a person skilled in the art that the recordings do not necessarily have to be created at the same time in order to be able to combine the image data to form a single image. Also, the image data do not necessarily have to be combined to form a single image, but instead may alternatively or additionally also be used or processed independently. Furthermore, individual images do not necessarily have to be processed; it is also possible for sequences of images of the two sensors to be processed to form a single sequence of images or else to form a single image. With the stereo images, 3D effects can be achieved by motion parallax, while a 2D sequence of images that gives the viewer a 3D impression is created from two or more 2D images and possible interpolation. In variants, instead of stereo images sequences of stereo images may also be used.

Further advantageous embodiments and combinations of features of the invention are obtained from the following detailed description and the patent claims as a whole.

### BRIEF DESCRIPTION OF THE DRAWINGS

In the drawings used for explaining the exemplary embodiment:

FIG. 1 shows a schematic representation of a plan view of a first embodiment of a device for examining an eye; and

FIG. 2 shows a schematic plan view of an optical part with three objectives.

In the figures, the same parts are in principle provided with same designations.

### WAYS OF IMPLEMENTING THE INVENTION

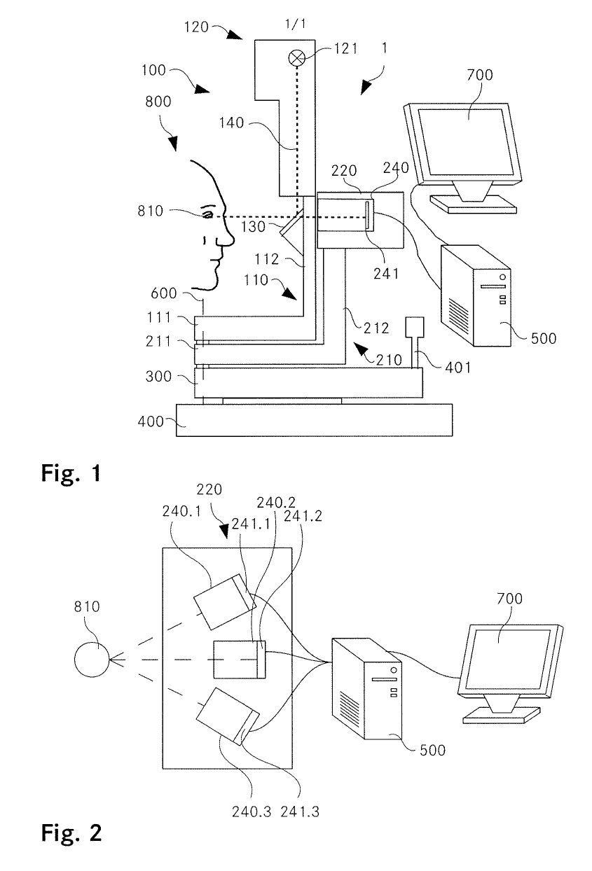

FIG. 1 shows a device 1 for examining an eye, formed as a slit lamp 1. The slit lamp 1 comprises a lighting unit 100, an optical part 220 and two objectives 240, which are mounted on a cross slide 300. The cross slide 300 itself is mounted on a base plate 400 which may be formed as a table top, and can be moved in the X, Y and Z directions. The slide 300 is controlled by means of an operator control unit 401, which is arranged on the same. The lighting unit 100 and the two objectives 240 can consequently be moved by means of the slide 300 and are also pivotably arranged independently of one another by means of a common axis of rotation 600.

The lighting unit 100 for the slit lighting comprises an L-shaped element 110 with a horizontal portion 111 and a vertical portion 112. In a distal region of the horizontal portion 111, the L-shaped element 110 comprises a vertically arranged axis of rotation 600. Arranged at the top of vertical portion is a lighting device 120, comprising a light source 121. The lighting unit 100 is made such that it can produce a defined strip of light, which can be projected onto an eye 810. On the inner side of the L-shaped element 110 there is on the vertical portion 112 a mirror 130, which is inclined from the vertical portion 112 at an angle of 45°. A light beam 140 produced by the light source 121 is directed vertically downward onto the mirror 130 arranged at an angle of 45°, and is directed from this mirror to the eye 810 of the patient 800. It is clear to a person skilled in the art that the lighting unit may also be arranged vertically under the mirror, while the mirror would be pivoted by an angle of 90°. Instead of a mirror, other optical elements with an analogous function may also be used. The lighting unit 100 may be pivotable about the axis 600 in a motorized manner or by hand. In a preferred embodiment, the lighting unit 100 is formed as a DLP projection device and is controlled purely electronically—but it is clear to a person skilled in the art that conventional mechanical devices may also be provided for producing the slit.

The image data are sent from the two image sensors 241 of the objectives 240 in the present embodiment to a computer 500. This may take place either by means of cables or else wirelessly in known ways. The computer 500 is represented in the present case as a separate unit. However, the computer 500 may also be component part of the device in the form of a computing unit. Similarly, in the present case a screen 700 is connected to the computer 500. This may also be a component part of the device. In particular, instead of the screen 700, other means known to a person skilled in the art for viewing digital image data may also be provided.

The rays of light coming back from the eye 810 in each case enter an optical part 220, which in the present case comprises two objectives 240. An objective 240 in each case comprises an image sensor 241, which may be of a one-part or multi-part form. The two objectives 240 are in the present case fixed objectives, which have a fixed magnification and moreover are arranged as fixed in the beam path. In the present case, the sensors are in each case a color sensor. Furthermore, in the present case, a focal plane can be set for the objectives 240; in particular. the focal plane can be changed over.

FIG. 2 shows a schematic representation of a second embodiment of an optical part 220, comprising three objectives 240.1, 240.2 and 240.3. The three objectives 240.1, 240.2 and 240.3 are in each case directed onto the eye 810, the focal planes of the three objectives 240.1, 240.2 and 240.3 intersecting in a common axis. The objectives 240.1 and 240.3 are arranged on the outside and enclose the objective 204.2, which is directed centrally onto the eye 810. With the objectives 240.1 and 240.3, stereoscopic images can be generated. The sensors 241.1, 241.2 and 241.3 of the objectives 240.1, 240.2 and 240.3 are connected to a computer 500. The sensors 241.1, 241.2 and 241.3 can be activated by means of a computing unit respectively of the computer 500 in such a way that images are recorded substantially at the same time. Moreover, the computing unit can be used for simultaneously activating the lighting unit 100, so that the sensors and the lighting unit 100 can be activated at the same time. In this way, defects in the recordings caused by reflexes can be largely masked out by means of image processing.

In a first variant, the objectives 240.1 and 240.3 are identically formed and comprise a color sensor 241.1 and 241.3, respectively, while the objective 240.2 comprises a black-and-white sensor 241.2 and is provided with a greater magnification. Consequently, with the two objectives 240.1 and 240.2 an overview image can be obtained. With the sensor 241.2 of the objective 240.2, details within the overview image can be reproduced with great resolution. The image material of the objective 240.2 can be colored on the basis of the data of the two objectives 240.1 and 240.3.

In a second variant, the objectives 240.1 and 240.3 are provided with great magnification and with black-and-white sensors 241.1 and 241.3, respectively, while the objective 240.2 is provided with a color sensor 241.2 and has a smaller magnification. Consequently, in the second variant the objective 240.2 is formed for creating an overview image, while with the two objectives 240.1 and 240.3 detailed stereoscopic recordings of the eye can be made in high resolution.

In a third variant, all of the sensors 241.1, 241.2 and 241.3 of the three objectives 240.1, 240.2 and 240.3 are formed as color sensors and, in a fourth variant, are formed as black-and-white sensors, the objective 240.2 having in each case a smaller magnification than the objectives 240.1 and 240.3.

In a fifth variant, the objective 240.2 comprises an IR sensor 241.2, while the objectives 240.1 and 240.3 in each case comprise a color sensor 241.1 and 241.3, respectively. In a sixth variant, the objective 240.2 comprises an IR sensor 241.2, while the objectives 240.1 and 240.3 comprise in each case a black-and-white sensor 241.1 and 241.3, respectively.

In a seventh variant, the individual objectives are arranged as fixed in relation to one another, while in an eighth variant at least one of the objectives in each case is pivotable about an axis. Preferably, at least the objectives 240.1 and 240.3 are pivotable about parallel axes in the vertical plane, while the objective 240.2 is arranged as fixed, in particular in an embodiment in which the objective 240.2 is formed for recording an overview image. Any number of other variants are known to a person skilled in the art.

It is also clear to a person skilled in the art that, instead of the precisely two or three objectives, more than three objectives may also be provided. The objectives may also be arranged in such a way that their optical axes run parallel or virtually parallel, in order to be able to cover a greater image region.

To sum up, it can be stated that the invention provides a device for examining an eye which is distinguished on the one hand by particularly precise and robust recordings and on the other hand by a simple and low-cost construction.

1-15. (canceled)

16. Device (1) for examining an eye, in particular a slit lamp, comprising an image recording unit (220) with at least one first sensor (241.1) in a first beam path and a second sensor (241.2) in a second beam path, characterized in that it also comprises in the first beam path a first objective with a first optical axis, which has a fixed magnification and is arranged as fixed in the first beam path.

17. Device (1) according to claim 16, characterized in that the first objective comprises the first sensor (241.1) and the device (1) also comprises a second objective with a second optical axis, the second objective comprising the second sensor (241.2).

18. Device (1) according to claim 16, characterized in that the first sensor (241.1) and the second sensor (241.2) differ in a recording spectrum.

19. Device (1) according to claim 18, characterized in that the first sensor (241.1) is formed as a color sensor and the second sensor (241.2) is formed as a black-and-white sensor or as an IR sensor.

20. Device (1) according to claim 16, characterized in that it has at least one third sensor (241.3) in a third beam path.

21. Device (1) according to claim 16, characterized in that a first magnification factor of the first objective is less than a second magnification factor of the second objective.

22. Device (1) according to claim 16, characterized in that an image recording region in a focal plane of the first beam path overlaps at least partially with an image recording region in a focal plane of the second beam path.

23. Device (1) according to claim 22, characterized in that the image recording region in the focal plane of the second beam path lies within the image recording region in the focal plane of the first beam path.

24. Device (1) according to claim 17, characterized in that an angle between the first optical axis and the second optical axis can be set.

25. Device (1) according to claim 24, characterized in that the first optical axis and the second optical axis can be set as parallel.

26. Device (1) according to claim 16, characterized in that a diaphragm and/or an optical filter are arranged in the first optical axis and in the second optical axis.

27. Method for determining image data with a device (1) according to claim 16, characterized by the following steps:
a. recording of image data of an object by the first sensor (241.1) and the second sensor (241.2);
b. processing image data of the first sensor (241.1) and the second sensor (241.2).

28. Method according to claim 27, characterized in that the image data of the object are recorded by the first sensor (241.1) and the second sensor (241.2) at the same time.

29. Method according to claim 27, characterized in that image data of a first sensor (241.1), formed as a black-and-white sensor with a first resolution, are computationally combined with image data of a second sensor (241.2), formed as a color sensor, to form a single colored image and/or to form a sequence of colored images.

30. Method according to claim 27, characterized in that the recording conditions of the first sensor (241.1) and the second sensor (241.2) differ.

31. Device (1) according to claim 17, characterized in that the second objective having a fixed magnification and being arranged as fixed in the second beam path.

32. Device (1) according to claim 20, characterized in that the third sensor (241.3) in the third beam path is comprised by the third objective

33. Device (1) according to claim 32, characterized in that the third objective having a fixed magnification.

34. Device (1) according to claim 32, characterized in that the second sensor (241.3) being formed as a color sensor.

35. Device (1) according to claim 32, characterized in that the third sensor (241.3) being formed as an IR sensor.

36. Device (1) according to claim 26, characterized in that the diaphragm and/or the optical filter are actuatable in a motorized manner.

37. Method according to claim 27, wherein the image of the object is recorded by the first sensor (241.1) and the second sensor (241.2) from different perspectives.

38. Method according to claim 27, wherein the image data of the first sensor (241.1) and the second sensor (241.2) are processed to form a single image and/or a sequence of images or respectively to form a single image segment and/or a sequence of image segments.

39. Method according to claim 29, wherein the second sensor (241.2) having a resolution that is in lower than the resolution of the first sensor (241.1)

40. Method according to claim 27, characterized in that the recording conditions of the first sensor (241.1) and the second sensor (241.2) differ in one of the following features:
a. exposure time;
b. diaphragm setting;
c. filter setting;
d. recording spectrum.
